(19) United States
(12) Patent Application Publication
(10) Pub. No.: US 2025/0312403 A1
(43) Pub. Date: Oct. 9, 2025

(54) ACNE CONTROL FORMULATIONS

(71) Applicant: STRITECH HOLDINGS PTY LTD, Ringwood, Victoria (AU)

(72) Inventor: Siu Ying Judy CHEUNG, Ringwood, Victoria (AU)

(21) Appl. No.: 18/866,726

(22) PCT Filed: May 17, 2022
(86) PCT No.: PCT/AU2022/050475

§ 371 Date: Nov. 18, 2024

(51) Int. Cl. A61K 36/185; A61K 31/07; A61K 31/197; A61K 31/4188; A61K 31/455; A61K 31/519; A61K 33/30; A61K 33/34; A61P 17/10

(52) U.S. Cl. CPC A61K 36/185; A61K 31/07; A61K 31/197; A61K 31/4188; A61K 31/455; A61K 31/519; A61K 33/30; A61K 33/34; A61P 17/10

(57) ABSTRACT

The present application relates to a composition comprising pantothenic acid, or a pharmaceutically acceptable derivative, salt or prodrug thereof; folic acid; nicotinamide; a source of copper; a source of zinc; and Urtica dioica. The composition can be used in a method for reducing, treating and/or preventing acne and/or related skin disorders. Additionally, the composition can be used in a method for reducing, treating and/or preventing the appearance of acne and/or related skin disorders on the skin.

### TECHNICAL FIELD

The present application relates to a composition comprising pantothenic acid, or a pharmaceutically acceptable derivative, salt or prodrug thereof; folic acid; nicotinamide; a source of copper; a source of zinc; and Urtica dioica. The composition can be used in a method for reducing, treating and/or preventing acne and/or related skin disorders. Additionally, the composition can be used in a method for reducing, treating and/or preventing the appearance of acne and/or related skin disorders on the skin.

### BACKGROUND

Acne is a common skin condition in which the skin pores become clogged leading to pimples and inflammation, often including infected abscesses. The development of sebum, being in the form of an excessive oil accumulation can sometimes dry, resulting in flaked skin and bacteria collecting in the skin pores and forming a comedo. The formation of a comedo blocks sebum from flowing from the hair follicles up to the pores, resulting in the formation of blackheads and sometimes whiteheads. Bacteria are then able to grow in the plugged pores and break down some of the fats in the sebum causing further irritation to the skin.

AU2013202114 discloses acne control compositions comprising pantothenic acid, or a pharmaceutically acceptable derivative, salt or prodrug thereof; folic acid; nicotinamide; a source of copper; and a source of zinc. The compositions may further include biotin, vitamin A, silicon and/or Vitex angus-castus.

The compositions of AU2013202114 were effective in lessening acne symptoms, but there is a possibility for further improvement. For example, the compositions of AU2013202114 further containing Vitex angus-castus had certain side effects. Some users complained that the compositions gave them headache. Some users did not like to use the compositions since Vitex angus-castus tends to lower testosterone level and thus, lowers sexual desires.

Therefore, formulating a more effective oral product with a plant-based ingredient other than Vitex angus-castus would help acne sufferers control acne symptoms with less side effects.

Therefore, there is a need and a demand for an improved oral product for the treatment and/or prevention of acne.

Any discussion of documents, acts, materials, devices, articles or the like which has been included in the present specification is not to be taken as an admission that any or all of these matters form part of the prior art base or were common general knowledge in the field relevant to the present invention as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### SUMMARY

In one aspect there is provided a composition for the treatment of acne comprising:
pantothenic acid, or a pharmaceutically acceptable derivative, salt or prodrug thereof;
folic acid;
nicotinamide;
a source of copper;
a source of zinc; and
Urtica dioica.

In another aspect there is provided a composition for the treatment of acne comprising the following:

| Component | Amount (weight %) |
|---|---|
| Biotin | 0.01-0.03 |
| Calcium pantothenate | 10-25 |
| Folic acid | 0.003-0.01 |
| Vitamin A acetate | 0.03-0.1 |
| Silicon | 0.30-0.80 |
| Copper gluconate | 0.05-0.20 |
| Nicotinamide | 5-10 |
| Zinc gluconate | 1.5-3.0 |
| Urtica dioica | 40-70 |

In another aspect there is provided a composition for the treatment of acne comprising the following:

| Component | Amount | |
|---|---|---|
| Biotin | 450-550 | μg |
| Calcium pantothenate | 500-600 | mg |
| Folic acid | 110-150 | μg |
| Vitamin A acetate | 150-250 | μg |
| Silicon | 10-15 | mg |
| Copper gluconate | 1-3 | mg |
| Nicotinamide | 150-200 | mg |
| Zinc gluconate | 50-60 | mg |
| Urtica dioica | 1,450-1,550 | mg |

In another aspect there is provided a composition for the treatment of acne comprising the following:

| Component | Amount (weight %) |
|---|---|
| Biotin | 0.03-0.06 |
| Calcium pantothenate | 35-65 |
| Folic acid | 0.005-0.02 |
| Vitamin A acetate | 0.01-0.05 |
| Silicon | 0.30-3.0 |
| Copper gluconate | 0.1-0.40 |
| Nicotinamide | 10-20 |
| Zinc gluconate | 3-7.0 |
| Urtica dioica root Extract | 5-15 |

In another aspect there is provided a composition for the treatment of acne comprising the following:

| Component | Amount | |
|---|---|---|
| Biotin | 450-550 | μg |
| Calcium pantothenate | 500-600 | mg |
| Folic acid | 110-150 | μg |
| Vitamin A acetate | 150-250 | μg |
| Silicon | 10-15 | mg |
| Copper gluconate | 1-3 | mg |
| Nicotinamide | 150-200 | mg |
| Zinc gluconate | 50-60 | mg |
| Urtica dioica root Extract | 50-150 | mg |

In yet another aspect there is provided a method of reducing, treating and/or preventing acne and/or related skin disorders comprising the administration of a composition to a subject in need of such treatment.

In yet another aspect there is provided a method of reducing, treating and/or preventing the appearance of acne and/or related skin disorders on the skin comprising the administration of a composition to a subject in need of such treatment.

In a further aspect there is provided the use of a composition in the manufacture of a medicament for the reduction, treatment and/or prevention of acne and/or related skin disorders.

In a further aspect there is provided the use of a composition in the manufacture of a medicament for the reduction, treatment and/or prevention of the appearance of acne and/or related skin disorders on the skin.

In yet a further aspect there is provided a composition when used for the reduction, treatment and/or prevention of the appearance of acne and/or related skin disorders on the skin.

### DESCRIPTION OF EMBODIMENTS

The present application provides an improved composition for the treatment of acne. The composition comprises
pantothenic acid, or a pharmaceutically acceptable derivative, salt or prodrug thereof;
folic acid;
nicotinamide;
a source of copper;
a source of zinc; and
Urtica dioica.

The inventors have surprisingly found that the composition of the present application is effective in the treatment of acne but does not appear to result in the side effects. The inventors have found that addition of Urtica dioica to the combination of components as described in the present composition can improve efficacy in the treatment of acne. Additionally, the present composition may reduce the dosage of the components as described in the present composition required for treating acne.

The compositions comprise pantothenic acid, or a pharmaceutically acceptable derivative, salt or prodrug thereof.

With respect to pantothenic acid, the term “pharmaceutically acceptable derivative” may include any pharmaceutically acceptable salt, hydrate, solvate, or prodrug, or any other compound which upon administration to a subject, is capable of providing (directly or indirectly) a compound of pantothenic acid or an active metabolite or residue thereof.

As used herein the term “salt” includes base addition, acid addition and quaternary salts. Suitable pharmaceutically acceptable salts include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, malic, citric, lactic, mucic, gluconic, benzoic, succinic, oxalic, phenylacetic, methanesulphonic, toluenesulphonic, benzenesulphonic, salicylic, sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, zinc, ammonium, alkylammonium such as salts formed from triethylamine, alkoxyammonium such as those formed with ethanolamine and salts formed from ethylenediamine, choline or amino acids such as arginine, lysine or histidine.

Basic nitrogen-containing groups may be quarternised with such agents as lower alkyl halide, such as methyl, ethyl, propyl, and butyl chlorides, bromides and iodides; dialkyl sulfates like dimethyl and diethyl sulfate, and others.

General information on types of pharmaceutically acceptable salts and their formation is known to those skilled in the art and is as described in general texts such as “Handbook of Pharmaceutical salts” P. H. Stahl, C. G. Wermuth, 1st edition, 2002, Wiley-VCH.

The term “solvate” is used herein to describe a molecular complex comprising the compound and a stoichiometric amount of one or more pharmaceutically acceptable solvent molecules, for example, ethanol. The term “hydrate” is employed when said solvent is water.

The term “prodrug”, is used herein to describe derivatives of compounds which may have little or no pharmacological activity themselves but which, when administered into or onto the body, are converted into compounds having the desired activity, for example, by hydrolytic cleavage. Further information on the use of prodrugs may be found in “Pro-drugs as Novel Delivery Systems”, Vol. 14, ACS Symposium Series (T. Higuchi and W. Stella) and Bioreversible Carriers in Drug Design, Pergamon Press, 1987 (ed. E. B. Roche, American Pharmaceutical Association).

Prodrugs can, for example, be produced by replacing appropriate functionalities present in a compound with certain moieties known to those skilled in the art as ‘pro-moieties’ as described, for example, in “Design of Prodrugs” by H. Bundgaard (Elsevier, 1985). For example, compounds having free amino, amido, hydroxy or carboxylic groups can be converted into prodrugs.

Prodrugs include compounds wherein an amino acid residue, or a polypeptide chain of two or more (eg, two, three or four) amino acid residues which are covalently joined to free amino, hydroxy and carboxylic acid groups. Prodrugs also include compounds wherein carbonates, carbamates, amides and alkyl esters are covalently bonded to amino, hydroxy and carboxylic acid groups. Prodrugs also include phosphate derivatives (such as acids, salts of acids, or esters) joined through a phosphorus oxygen bond to a free hydroxyl group.

Examples of pantothenic acid, a pharmaceutically acceptable derivative, salt or prodrug thereof, suitable for use in the present compositions include, but are not limited to, pantothenic acid, calcium pantothenate and pantothenol. In a specific embodiment, the pantothenic acid, a pharmaceutically acceptable derivative, salt or prodrug thereof, is calcium pantothenate.

The compositions can comprise the pantothenic acid, or a pharmaceutically acceptable derivative, salt or prodrug thereof, in an amount sufficient to provide an amount of pantothenic acid of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, or 60% by weight of the composition, or a range comprising any of two of those integers. In one embodiment, the compositions comprise an amount of pantothenic acid of from about 1% to about 60% by weight of the composition. In another embodiment, the compositions comprise an amount of pantothenic acid of from about 15% to about 30% by weight of the composition. In yet another embodiment, the compositions comprise an amount of pantothenic acid of from about 5% to about 15% by weight of the composition . . . . In yet another embodiment, the compositions comprise an amount of pantothenic acid of from about 10% to about 20% by weight of the composition. In yet another embodiment, the compositions comprise an amount of pantothenic acid of from about 35% to about 45% by weight of the composition.

The compositions can comprise the pantothenic acid, or a pharmaceutically acceptable derivative, salt or prodrug thereof, in an amount sufficient to provide an amount of pantothenic acid of about 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690 or 700 mg, or a range comprising any of two of those integers. In one embodiment, the compositions comprise an amount of pantothenic acid of from about 100 mg to about 700 mg. In another embodiment, the compositions comprise an amount of pantothenic acid of from about 100 mg to about 250 mg. In yet another embodiment, the compositions comprise an amount of pantothenic acid of from about 350 mg to about 700 mg.

The compositions can comprise folic acid. Folic acid is also known as folate, vitamin M, vitamin B₉, vitamin B_{c} (or folacin), pteroyl-L-glutamic acid, pteroyl-L-glutamate, and pteroylmonoglutamic acid.

The compositions can comprise folic acid in an amount of about 0.001, 0.002, 0.003, 0.004, 0.005, 0.006, 0.007, 0.008, 0.009, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095 or 0.1% by weight of the composition, or a range comprising any of two of those integers. In one embodiment, the compositions comprise folic acid in an amount of from about 0.001% to about 0.1% by weight of the composition. In another embodiment, the compositions comprise folic acid in an amount of from about 0.001% to about 0.02% by weight of the composition.

The compositions can comprise folic acid in an amount of about 50, 55, 60, 65, 70, 75, 80, 85, 90, 95, 100, 105, 110, 115, 120, 125, 130, 135, 140, 145, 150, 155, 160, 165, 170, 175, 180, 185, 190, 195 or 200 μg, or a range comprising any of two of those integers. In one embodiment, the compositions comprise folic acid in an amount of from about 100 μg to about 200 μg. In another embodiment, the compositions comprise folic acid in an amount of from about 80 μg to about 150 μg. In yet another embodiment, the compositions comprise folic acid in an amount of from about 110 μg to about 200 μg.

The compositions comprise nicotinamide. Nicotinamide is also known as niacinamide and nicotinic acid amide and is the amide of nicotinic acid (vitamin B3/niacin).

The compositions can comprise nicotinamide in an amount of about, 1, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 19, 20, 21, or 22% by weight of the composition, or a range comprising any of two of those integers. In one embodiment, the compositions comprise nicotinamide in an amount of from about 5% to about 18% by weight of the composition. In another embodiment, the compositions comprise nicotinamide in an amount of from about 3% to about 12% by weight of the composition. In another embodiment, the compositions comprise nicotinamide in an amount of from about 5% to about 10% by weight of the composition. In another embodiment, the compositions comprise nicotinamide in an amount of from about 10% to about 15% by weight of the composition.

The compositions can comprise nicotinamide in an amount of about 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245 or 250 mg, or a range comprising any of two of those integers. In one embodiment, the compositions comprise nicotinamide in an amount of from about 70 mg to about 250 mg. In another embodiment, the compositions comprise nicotinamide in an amount of from about 150 mg to about 200 mg.

The compositions comprise a source of copper. Examples of suitable sources of copper include, but are not limited to, copper gluconate, copper sulphate, copper acetate and copper citrate. In one embodiment the source of copper is copper gluconate.

The compositions can comprise copper in an amount of about 0.001, 0.002, 0.003, 0.004, 0.005, 0.006, 0.007, 0.008, 0.009, 0.01, 0.015, 0.02, 0.025, 0.03, 0.035, 0.04, 0.045, 0.05, 0.06, 0.07, 0.08, 0.09, 0.10, 0.11, 0.12, 0.13, 0.14, 0.15, 0.16, 0.17, 0.18, 0.19, or 0.20 by weight of the composition, or a range comprising any of two of those integers. In one embodiment, the compositions comprise copper in an amount of from about 0.001% to about 0.05% by weight of the composition. In another embodiment, the compositions comprise copper in an amount of from about 0.005% to about 0.03% by weight of the composition.

The compositions can comprise copper in an amount of about 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, and 500 μg or a range comprising any of two of those integers. In one embodiment, the compositions comprise copper in an amount of from about 200 μg to about 450 μg. In another embodiment, the compositions comprise copper in an amount of about 350 μg to 400 μg.

The compositions can comprise a source of zinc. Examples of suitable sources of zinc include, but are not limited to, zinc gluconate, zinc sulphate, zinc acetate and zinc citrate. In one embodiment the source of zinc is zinc gluconate.

The compositions can comprise zinc in an amount of about 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.05, 1.10, 1.15, 1.20, 1.25, 1.30, 1.35, 1.40, 1.45, 1.50, 1.55, 1.60, 1.65, 1.70, 1.75, 1.80, 1.85, 1.90, 1.95 or 2.0% by weight of the composition, or a range comprising any of two of those integers. In one embodiment, the compositions comprise zinc in an amount of from about 0.05% to about 2% by weight of the composition. In another embodiment, the compositions comprise zinc in an amount of from about 0.1% to about 1.5% by weight of the composition.

The compositions can comprise zinc in an amount of from about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5 or 20 mg or a range comprising any of two of those integers. In one embodiment, the compositions comprise zinc in an amount of from about 1 mg to about 20 mg. In another embodiment, the compositions comprise zinc in an amount of from about 5 mg to about 15 mg.

The compositions comprise Urtica dioica. Urtica dioica, which is commonly known as stinging nettle or simply known as nettle, is an herbaceous perennial flowering plant in the family Urticaceae. In one embodiment, Urtica dioica may be an extract of Urtica dioica. In one embodiment, the Urtica dioica extract may be prepared by contacting Urtica dioica with one or more of polar solvents. In one embodiment, the Urtica dioica extract may be prepared by contacting Urtica dioica with a mixture of an alcohol such as methanol or ethanol and water. In one embodiment, the Urtica dioica extract may be prepared by contacting Urtica dioica with a mixture of methanol and water. In one embodiment, the Urtica dioica extract may be prepared by contacting Urtica dioica with a mixture of methanol and water in the ratio of 10:1, 11:1, 12:1, 13:1, 14:1, 15:1, 16:1, 17:1, 18:1, 19:1, or 20:1. In one embodiment, the Urtica dioica extract may be prepared by contacting Urtica dioica with a mixture of 30% methanol and water in the ratio of 16:1. In one embodiment the Urtica dioica extract may be Urtica dioica root extract. In one embodiment the Urtica dioica root extract may be a dry concentrate.

The compositions can comprise Urtica dioica in an amount of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70% by weight of the composition, or a range comprising any of two of those integers. In one embodiment, the compositions comprise Urtica dioica in an amount of from about 10% to about 70% by weight of the composition. In another embodiment, the compositions comprise Urtica dioica in an amount of from about 30% to about 70% by weight of the composition. In another embodiment, the compositions comprise Urtica dioica in an amount of from about 40% to about 70% by weight of the composition.

The compositions can comprise Urtica dioica in an amount of from about 100, 200, 300, 400, 500, 600, 700, 800, 900, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950 and 3000 mg or a range comprising any of two of those integers. In one embodiment, the compositions comprise Urtica dioica in an amount of from about 1200 mg to about 1800 mg. In another embodiment, the compositions comprise Urtica dioica in an amount of from about 1400 mg to about 1600 mg. In another embodiment, the compositions comprise Urtica dioica in an amount of from about 1450 mg to about 1550 mg.

The compositions can comprise Urtica dioica root extract in an amount of about 1, 2, 3, 4, 5, 6, 7, 8, 9, 10, 11, 12, 13, 14, 15, 16, 17, 18, 19, or 20% by weight of the composition, or a range comprising any of two of those integers. In one embodiment, the compositions comprise Urtica dioica root extract in an amount of from about 3% to about 7% by weight of the composition. In another embodiment, the compositions comprise Urtica dioica root extract in an amount of from about 5% to about 10% by weight of the composition. In another embodiment, the compositions comprise Urtica dioica root extract in an amount of from about 10% to about 20% by weight of the composition. In another embodiment, the compositions comprise Urtica dioica root extract in an amount of from about 5% to about 15% by weight of the composition.

The compositions can comprise Urtica dioica root extract in an amount of from about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450,460, 470, 480, 490 or 500 mg, or a range comprising any of two of those integers. In one embodiment, the compositions comprise Urtica dioica root extract in an amount of from about 50 mg to about 150 mg. In another embodiment, the compositions comprise Urtica dioica root extract in an amount of from about 150 mg to about 250 mg. In another embodiment, the compositions comprise Urtica dioica root extract in an amount of from about 80 mg to about 120 mg.

In one embodiment, Urtica dioica in the compositions is in a form of Urtica dioica root extract. In one embodiment, from about 80 mg to 120 mg of Urtica dioica root extract may be equivalent to from about 1300 mg to 1800 mg of dried Urtica dioica root. In one embodiment, from about 90 mg to 110 mg of Urtica dioica root extract may be equivalent to from about 1400 mg to 1700 mg of dried Urtica dioica root. In one embodiment, from about 90 mg to 95 mg of Urtica dioica root extract may be equivalent to from about 1450 mg to 1550 mg of dried Urtica dioica root. In one embodiment, about 93.75 mg of Urtica dioica root extract may be equivalent to about 1500 mg of dried Urtica dioica root.

In one aspect, the compositions comprise biotin. Biotin is also known as vitamin H or coenzyme R. The compositions may comprise biotin in an amount of 0.001, 0.002, 0.003, 0.004, 0.005, 0.006, 0.007, 0.008, 0.009, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095 and 0.1% by weight of the composition, or a range comprising any of two of those integers. In one embodiment, the compositions comprise biotin in an amount of from about 0.001% to about 0.1% by weight of the composition. In another embodiment, the compositions comprise biotin in an amount of from about 0.01% to about 0.05% by weight of the composition.

The compositions may comprise biotin in an amount of 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 525, 530, 535, 540, 545 and 550 μg or a range comprising any of two of those integers. In one embodiment, the compositions comprise biotin in an amount of from about 250 μg to about 550 μg. In another embodiment, the compositions comprise biotin in an amount of from about 450 μg to about 550 μg.

In one aspect, the compositions comprise a form of vitamin A. Examples of forms of vitamin A suitable for use in the compositions include, but are not limited to, vitamin A acetate, retinol, retinoic acid, retinyl acetate, retinyl palmitate and beta-carotene. In one embodiment, the form of vitamin A is Vitamin A acetate.

The compositions may comprise a form of vitamin A in an amount of 0.001, 0.002, 0.003, 0.004, 0.004, 0.005, 0.005, 0.006, 0.007, 0.008, 0.009, 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.15, 0.2, 0.25, 0.3, 0.35, 0.4, 0.45 and 0.5% by weight of the composition, or a range comprising any of two of those integers. In one embodiment, the compositions comprise a form of vitamin A in an amount of from about 0.001% to about 0.5% by weight of the composition. In another embodiment, the compositions comprise a form of vitamin A in an amount of from about 0.01% to about 0.1% by weight of the composition.

The compositions may comprise a form of vitamin A in an amount of 50, 60, 70, 80, 90, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 1110, 1125, 1150, 1175 and 1200 μg or a range comprising any of two of those integers. In one embodiment, the compositions comprise a form of vitamin A in an amount of from about 50 μg to about 1200 μg. In another embodiment, the compositions comprise a form of vitamin A in an amount of from about 700 μg to about 1100 μg. In another embodiment, the compositions comprise a form of vitamin A in an amount of from about 150 μg to about 300 μg.

In one aspect, the compositions comprise silicon. Examples of forms of silicon suitable for use in the compositions include, but are not limited to, silicon dioxide, orthosilicic acid, choline-stabilized orthosilicic acid and colloidal anhydrous silica. In one embodiment, the form of silicon is colloidal anhydrous silica.

The compositions may comprise silicon in an amount of 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9 and 2% by weight of the composition, or a range comprising any of two of those integers. In one embodiment, the compositions comprise silicon in an amount of from about 0.01% to about 2% by weight of the composition. In another embodiment, the compositions comprise silicon in an amount of from about 0.5% to about 1.5% by weight of the composition.

The compositions may comprise silicon in an amount of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 mg or a range comprising any of two of those integers. In one embodiment, the compositions comprise silicon in an amount of from about 1 mg to about 50 mg. In another embodiment, the compositions comprise silicon in an amount of from about 5 mg to about 15 mg.

Further optional excipients may also be included in the compositions. Such optional excipients include, but are not limited to, antiadherents, binders, coatings, disintegrants, fillers, flavours, colours, lubricants, glidants, sorbents, preservatives and sweeteners. Examples of binders include, but are not limited to, sugars (such as mono- and disaccharides, polysaccharides, and their derivatives including starches, cellulose such as microcrystalline cellulose and cellulose ethers such as hydroxypropyl cellulose), sugar alcohols (such as xylitol, sorbitol and maltitol), proteins (such as gelatin) and polymers (such as PVP, crospovidone, polyethylene glycols and polypropyleneglycols). Examples of coatings include, but are not limited to, cellulose ethers (such as hydroxypropoyl methyl cellulose), synthetic polymers, shellac, gelatin, polysaccharides and coating systems available under the tradename Opadry®. Examples of disintegrants include, but are not limited to, starches (such as sodium starch glycolate), and cross-linked polymers, (such as crospovidone and croscarmellose sodium). Examples of fillers include, but are not limited to, fats and oils, calcium phosphate, dibasic calcium phosphate, lactose, sucrose, glucose, mannitol, sorbitol, calcium carbonate and magnesium stearate. Examples of lubricants include, but are not limited to, talc, silica, fats (such as vegetable stearin, magnesium stearate and stearic acid). Examples of glidants include, but are not limited to, silica, talc, and magnesium carbonate. Examples of sorbents include, but are not limited to, fatty acids, waxes, shellac, plastics, and plant fibers. Examples of preservatives include, but are not limited to, antioxidants, the amino acids cysteine and methionine, citric acid and sodium citrate, and parabens (such as methyl paraben and propyl paraben). In one embodiment, the optional excipients include, but are not limited to, calcium phosphate, calcium hydrogen phosphate, microcrystalline cellulose, crospovidone, magnesium stearate and Opadry® coatings. In some embodiment excipients include calcium hydrogen phosphate dehydrate, Carnauba Wax, crospovidone, lecithin, macrogol 3000, magnesium stearate, microcrystalline cellulose, polyvinyl alcohol, povidone, purified talc, and/or titanium dioxide.

In one aspect, the compositions comprising pantothenic acid, or a pharmaceutically acceptable derivative, salt or prodrug thereof, folic acid; nicotinamide; a source of copper; a source of zinc; and Urtica dioica are in a dosage form selected from the group consisting of, but not limited to, a tablet, caplet, capsule, gel cap, pellet and granule. In one embodiment, the dosage form is a caplet. In another embodiment, the dosage form is a tablet. In a specific embodiment, the tablet weighs from about 500 mg to about 3000 mg. In a specific embodiment, the tablet weighs from about 700 mg to about 3000 mg. In a specific embodiment, the tablet weighs from about 1000 mg to about 2000 mg. In a specific embodiment, the tablet weighs from about 1000 mg to about 2500 mg. In a specific embodiment, the tablet weighs from about 2000 mg to about 3000 mg. In another embodiment, the dosage form is a coated caplet or a coated tablet. In yet another embodiment, the dosage form is an oral dosage form.

In one aspect, the compositions comprising pantothenic acid, or a pharmaceutically acceptable derivative, salt or prodrug thereof; folic acid; nicotinamide; a source of copper; a source of zinc; and Urtica dioica are used in a method for reducing, treating and/or preventing acne and/or related skin disorders. Acne related skin disorders includes other follicular disorders and acne-like skin conditions which would be known to a skilled person.

The method for reducing, treating and/or preventing acne and/or related skin disorders comprises the administration of a composition to a subject in need of such treatment. In one embodiment, the method for reducing, treating and/or preventing acne and/or related skin disorders on the skin comprises administration of the composition to the subject orally. In another embodiment, administration of the composition provides to the subject Urtica dioica in an amount of between about 0.75 g and about 7.5 g per day. In a specific embodiment, administration of the composition provides to the subject Urtica dioica in an amount of about 0.75 g per day. In a specific embodiment, administration of the composition provides to the subject Urtica dioica in an amount of about 1.5 g per day. In a specific embodiment, administration of the composition provides to the subject Urtica dioica in an amount of about 2.25 g per day. In a specific embodiment, administration of the composition provides to the subject Urtica dioica in an amount of about 3 g per day. In a specific embodiment, administration of the composition provides to the subject Urtica dioica in an amount of about 3.75 g per day. In a specific embodiment, administration of the composition provides to the subject Urtica dioica in an amount of about 4.5 g per day. In a specific embodiment, administration of the composition provides to the subject Urtica dioica in an amount of about 5.25 g per day. In a specific embodiment, administration of the composition provides to the subject Urtica dioica in an amount of about 6 g per day. In a specific embodiment, administration of the composition provides to the subject Urtica dioica in an amount of about 7.5 g per day. In a further embodiment, administration of the composition provides to the subject Urtica dioica in an amount of between about 3 g and about 6 g per day.

The compositions may be used in a method relating to the following types of acne: acne vulgaris (common acne, including blackheads and whiteheads), cystic acne, nodulocystic acne, acne excorieé, acne fulminans, infantile acne (acne in babies), acne in pregnancy, acne due to medicines, adult acne, acne scarring, chloracne, comedonal acne and pyoderma faciale. Acne related skin disorders include skin conditions that appear similar to acne. Such skin conditions include, but are not limited to, acne keloidalis nuchae (acne cheloidalis), acne necrotica (scalp folliculitis), acne urticata (itchy spots), comedone naevus, cysts, disseminate and recurrent infundibulofolliculitis, folliculitis, gram negative folliculitis, hidradenitis suppurativa, hot tub (spa pool) folliculitis, keratosis pilaris, lichen spinulosus, miliaria (sweat rash), neonatal cephalic pustulosis, oil folliculitis, perioral dermatitis (muzzle rash), pityrosporum folliculitis, pseudofolliculitis barbae, pustular tinea, rosacea, sebaceous, hyperplasia, solar (senile) comedones, seborrhoea (oily skin), staphylococcal folliculitis, steatocystoma multiplex, steroid acne, steroid rosacea and trichostasis spinulosa.

In another aspect, the compositions comprising pantothenic acid, or a pharmaceutically acceptable derivative, salt or prodrug thereof, folic acid; nicotinamide; a source of copper; a source of zinc; and Urtica dioica are used in a method for reducing, treating and/or preventing the appearance of acne and/or related skin disorders on the skin.

The method for reducing, treating and/or preventing the appearance of acne and/or related skin disorders on the skin comprises the administration of a composition to a subject in need of such treatment. In one embodiment, the method for reducing, treating and/or preventing the appearance of acne and/or related skin disorders on the skin comprises administration of the composition to the subject orally. In another embodiment, administration of the composition provides to the subject Urtica dioica in an amount of between about 1.5 g and about 7.5 g per day. In a specific embodiment, administration of the composition provides to the subject Urtica dioica in an amount of about 0.75 g per day. In a specific embodiment, administration of the composition provides to the subject Urtica dioica in an amount of about 1.5 g per day. In a specific embodiment, administration of the composition provides to the subject Urtica dioica in an amount of about 2.25 g per day. In a specific embodiment, administration of the composition provides to the subject Urtica dioica in an amount of about 3 g per day. In a specific embodiment, administration of the composition provides to the subject Urtica dioica in an amount of about 3.75 g per day. In a specific embodiment, administration of the composition provides to the subject Urtica dioica in an amount of about 4.5 g per day. In a specific embodiment, administration of the composition provides to the subject Urtica dioica in an amount of about 5.25 g per day. In a specific embodiment, administration of the composition provides to the subject Urtica dioica in an amount of about 6 g per day. In a specific embodiment, administration of the composition provides to the subject Urtica dioica in an amount of about 7.5 g per day. In a further embodiment, administration of the composition provides to the subject Urtica dioica in an amount of between about 3 g and about 6 g per day.

In one aspect, the composition comprising pantothenic acid, or a pharmaceutically acceptable derivative, salt or prodrug thereof; folic acid; nicotinamide; a source of copper; a source of zinc; and Urtica dioica are used for the manufacture of a medicament for the reduction, treatment and/or prevention of acne and/or related skin disorders.

In another aspect, the composition comprising pantothenic acid, or a pharmaceutically acceptable derivative, salt or prodrug thereof, folic acid; nicotinamide; a source of copper; a source of zinc; and Urtica dioica are used for the manufacture of a medicament for the reduction, treatment and/or prevention of the appearance of acne and/or related skin disorders on the skin.

### EXAMPLES

### Material and Methods

The components used in the formulations were sourced from Lipa Pharmaceuticals Limited based in Minto, New South Wales, Australia.

The starting materials used in the formulations were verified and assessed to be compliant to the requirements for the residual solvents. Annual verification testing was conducted to ensure compliance and was recorded as part of the Product Quality Review. Solvents that were used as part of the manufacturing process are listed with their respective limits below.
Ethanol: Ethanol Not more than 5000 ppm or NMT 50 mg/Day

The starting materials used in this formula have been assessed to allow the finished product to meet the below limits of Elemental impurities. Annual verification testing was conducted to ensure compliance and was recorded as part of the Product Quality review.
Arsenic: Not more than 2.0 ppm
Cadmium: Not more than 0.7 ppm
Lead: Not more than 0.7 ppm
Mercury: Not more than 0.3 ppm

### Formulating Process

The film coated tablets disclosed in the Examples section were prepared according to the following processes. Raw Materials were tested to ensure that the materials did meet specification before being released to production of manufacture. The raw materials were weighed and dispensed to the manufacturing site. The raw materials were mixed and blended in a double cone blender. The mixed and blended materials were compressed by utilising oval tooling with break bar to afford tablets. Each tablet was coated with coating materials comprising Opadry II complete film coating system and Carnauba Wax. The manufactured tablets were inspected and packaged.

### Formulations

Table 1 provides Example Composition 1 comprising pantothenic acid, or a pharmaceutically acceptable derivative, salt or prodrug thereof; folic acid; nicotinamide; a source of copper; and a source of zinc. The weight % in Table 1 is calculated based on weight % of the total amount of the active ingredients listed in Table 1. Example Composition 1 is a film coated tablet for oral administration.

TABLE 1
Example Composition 1.

| Component | Amount (weight %) | Amount/unit | |
|---|---|---|---|
| Biotin | 0.049 | 330 | μg |
| Calcium pantothenate | 74.571 | 500 | mg |
| Folic acid | 0.012 | 80 | μg |
| Vitamin A acetate | 0.020 | 135.7 | μg |
| Silicon | 0.746 | 5 | mg |
| Copper gluconate | 0.268 | 1.8 | mg |
| Nicotinamide | 18.643 | 125 | mg |
| Zinc gluconate | 5.711 | 38.29 | mg |

The Example Composition 1 further comprises the following excipients and coating listed in Table 2.

TABLE 2
Excipients and coating of Example Composition 1.

| | Amount (mg)/unit | Function |
|---|---|---|
| Excipients | | |
| Povidone | 40.00 | Binder |
| Microcrystalline cellulose | 298.36 | Filler |
| Calcium hydrogen phosphate dehydrate | 0.50 | Filler |
| Crospovidone | 0.50 | Disintegrant |
| Purified talc | 0.20 | Anti-caking agent |
| Magnesium stearate | 16.00 | Lubricant |
| Coating | | |
| Opadry II complete film coating system | 27.50 | Colourant |
| 85G58921 white | | |
| Carnauba Wax | 0.20 | Polishing agent |

Table 3 provides Example Composition 2 comprising pantothenic acid, or a pharmaceutically acceptable derivative, salt or prodrug thereof; folic acid; nicotinamide; a source of copper; a source of zinc; and Urtica dioica (Nettle) root extract. The weight % in Table 3 is calculated based on weight % of the total amount of the active ingredients listed in Table 3. The Example Composition 2 is a film coated tablet for oral administration.

TABLE 3
Example Composition 2.

| Component | Amount (weight %) | Amount/unit | |
|---|---|---|---|
| Biotin | 0.055 | 500 | μg |
| Calcium pantothenate | 59.7 | 545.9 | mg |
| Folic acid | 0.014 | 125 | μg |
| Vitamin A acetate | 0.023 | 206.4 | μg |
| Equivalent to vitamin A 175RE | | | |
| Colloidal anhydrous silica | 2.9 | 26.74 | mg |
| equivalent to silicon 12.5 mg | | | |
| Copper gluconate | 0.293 | 2.68 | mg |
| Equivalent to copper 375 μg | | | |
| Nicotinamide | 20.49 | 187.5 | mg |
| Zinc gluconate | 6.28 | 57.43 | mg |
| equivalent to zinc 7.5 mg | | | |
| Urtica dioica (Nettle) root extract | 10.25 | 93.75 | mg |

dry concentrate
(16:1 in 30% Methanol:Water)
equivalent. to Urtica Dioica Root
dry 1500 mg

The Example Composition 2 further comprise the following excipients and coating listed in Table 4.

TABLE 4
Excipients and coating of Example Composition 2.

| | Amount (mg)/unit | Function |
|---|---|---|
| Excipients | | |
| Povidone | 45.00 | Binder |
| Microcrystalline cellulose | 346.56 | Filler |
| Calcium hydrogen phosphate dehydrate | 1.00 | Filler |
| Crospovidone | 10.00 | Disintegrant |
| Purified talc | 0.20 | Anti-caking agent |
| Magnesium stearate | 16.00 | Lubricant |
| Coating | | |
| Opadry II complete film coating system | 49.50 | Colourant |
| 85G58921 white | | |
| Carnauba Wax | 0.20 | Polishing agent |

The following provides examples of the use of Example Composition 1 and Example Composition 2 in methods for treating acne.

### Example 1

40 subjects suffering from acne were administered with Example composition 1, according to the regime of 2 tablets, three times a day for the period shown in Table 5. The subjects administered with Example composition 1 observed that their acne symptoms are lessened as shown in Table 5.

TABLE 5
Efficacy survey data for Example Composition 1

| Subject | | 1-4 | 4-6 | 8 | 3 | 6 | 1 | 1 |
|---|---|---|---|---|---|---|---|---|
| No. | Type of Acne | weeks | weeks | weeks | months | months | year | year+ |
| 1 | mild/occasional | | | | | I | S | C |
| 2 | moderate | I | S | C | | | | |
| 3 | mild/occasional | I | S, C | | | | | |
| 4 | mild/occasional | I, S | | | C | | | |
| 5 | mild/occasional | I | | | S, C | | | |
| 6 | mild/occasional | I | | S | | C | | |
| 7 | moderate | I | S, C | | | | | |
| 8 | mild/occasional | | | I | S, C | | | |
| 9 | mild/occasional | I | | S | C | | | |
| 10 | moderate | | | I, S | C | | | |
| 11 | moderate | I | | S | | C | | |
| 12 | mild/occasional | I | | S, C | | | | |
| 13 | moderate | | | | S, C | I | | |
| 14 | moderate | I | | | S, C | | C | |
| 15 | moderate | | | I | | S | | C |
| 16 | moderate | I | | | S, C | | C | |
| 17 | moderate | I, S | | | C | | | |
| 18 | moderate | | | I | S, C | | C | |
| 19 | mild/occasional | I | | S | C | | | |
| 20 | moderate | I | | S | | C | | |
| 21 | moderate | I | | S | | | C | |
| 22 | mild/occasional | | | I | | S | C | |
| 23 | severe | | | S | I | C | | |
| 24 | moderate | | | | I | S | C | |
| 25 | moderate | I | | S | C | | | |
| 26 | moderate | | | I | S | C | | |
| 27 | moderate | I | | S | C | | | |
| 28 | severe | I | | S, C | | | | |
| 29 | moderate | | | | I, S | | C | |
| 30 | severe | I | | | S | C | | |
| 31 | severe | | | | I | S | | C |
| 32 | mild/occasional | I | | | S | | C | |
| 33 | moderate | | | I, S | C | | | |
| 34 | moderate | | | | I, S | | | C |
| 35 | severe | | | I | S | | | C |
| 36 | mild/occasional | | | I | S, C | | | |
| 37 | mild/occasional | | | | | I | S | C |
| 38 | mild/occasional | I | | S | C | | | |
| 39 | mild/occasional | ISC | | | | | | |
| 40 | mild/occasional | | | I | S | C | | |

I = improvement
S: significant improvement
C: completely clear

Table 5 shows the efficacy of the Example Composition 1 during the period or 1-4 weeks, 4-6 weeks, 8 weeks, 3 months, 6 months, 1 year, or over 1 year from the administration of the Example Composition 1. The subjects administered with the Example Composition 1 observed a reduction in the appearance of their acne.

At 1-4 weeks 22 out of 40 subjects showed improvement in their acne symptoms. Thus, 55% of the subjects showed improvement within 4 weeks from administering the Example Composition 1.

Up to 8 weeks 32 out of 40 subjects showed improvement in their acne symptoms. Thus, 80% of the subjects showed improvement within 8 weeks from administering the Example Composition 1.

Up to 3 months 37 out of 40 subjects showed improvement or significant improvement in their acne symptoms. Thus, 92.5% of the subjects showed improvement or significant improvement within 3 months from administering the Example Composition 1.

### Example 2

42 subjects suffering from acne were administered with Example Composition 2, according to the regime of 2 tablets, two times a day for the period shown in Table 6. A few subjects were administered with 1 tablet per day or 2 tablets per day as shown in Table 6. The subjects observed that their acne symptoms are significantly lessened as shown in Table 6.

TABLE 6
Efficacy survey data for the Example composition 2

| | | | | | | | |
|---|---|---|---|---|---|---|---|
| Subject | Type of | 1-4 | 4-6 | 8 | 3 | 6 | 1 |
| No. | Acne | weeks | weeks | weeks | months | months | year |

| | | | | | | |
|---|---|---|---|---|---|---|
| 1 | hormonal | I | | S-9 | C-15 | |
| | Acne | | | weeks | weeks | |
| 2 | adult acne | I | S | | | |
| 3 | adult acne | I | | S | | |
| 4 | adult acne | I, S | | | | |
| 5 | teenage and | I | | S | | |
| | adult Acne | | | | | |
| 6 | adult acne | | I | | S | |
| | and body | | | | | |
| | acne | | | | | |
| 7 | adult acne | I | S | | | |
| 8 | Mask acne | I, S | C | | | |
| 9 | adult acne | I, S, C | | | | |
| 10 | adult acne | I | | S | | |
| 11 | adult acne | I | | S | | |
| 12 | adult acne | I, S | | | | |
| 13 | adult acne | | I | | S | |
| 14 | adult acne | I, S, C | | | | |
| 15 | adult acne | I | S | | | |
| 16 | Teenage Acne | I | | | S | |
| 17 | Adult acne | I, S | | | | |
| 18 | Adult Acne | I, S | | | | |
| 19 | adult Acne | I | | | | |
| 20 | adult acne | I | | | | |
| 21 | adult acne | I | | S | | C |
| 22 | adult acne | I, S | | | | |
| 23 | adult acne | I | | | | |
| 24 | adult acne | I, S | | | | |
| 25 | teenage acne | I | S | | C | |
| 26 | adult acne | I, S | | | | |
| 27 | adult acne | I, S | | | | |
| 28 | adult acne | I | | S | | |
| 29 | adult acne | I, S | | | | |
| 30 | adult acne | I-2 | S | | C | |
| | | weeks | | | | |
| 31 | adult acne, | I | | S | | |
| | acne scar and | | | | | |
| | hormonal | | | | | |
| | acne | | | | | |
| 32 | adult acne | I-2 | | S-9 | | |
| | and acne scar | weeks | | weeks | | |
| 33 | Teenage Acne | I, S-1 | | | | |
| | | week | | | | |
| 34 | Hormonal | I, S-4 | | | C | |
| | acne (1 tablet | week | | | | |
| | every | | | | | |
| | morning) | | | | | |
| 35 | Teenage Acne | I, S-3 | | | | |
| | (2 tablets, | weeks | | | | |
| | twice daily) | | | | | |
| 36 | Blackheads, | I-2 | | | | |
| | skin | weeks | | | | |
| | Congestions | | | | | |
| 37 | Adult/ | I, S-4 | | | | |
| | Hormonal | weeks | | | | |
| 38 | Adult acne (2 | I | | S | | C |
| | tablets twice | | | | | |
| | a day for 6 | | | | | |
| | months. Then | | | | | |
| | 1 tab twice a | | | | | |
| | day | | | | | |
| | thereafter) | | | | | |
| 39 | Hormonal | I | | S | | |
| | Acne (1 | | | | | |
| | tablet, 2 | | | | | |
| | times a day) | | | | | |
| 40 | Hormonal | I, S-4 | | | | |
| | Acne (1 | weeks | | | | |
| | tablet, 2 | | | | | |
| | times a day) | | | | | |
| 41 | hormonal | I, S-3 | | | | |
| | Acne (1 | weeks | | | | |
| | tablet a day) | | | | | |
| 42 | Persistent | I, S-4 | | | | |
| | Acne (2 | weeks | | | | |
| | tablets, 2 | | | | | |
| | times a day) | | | | | |

I = improvement
S: significant improvement
C: completely clear

Table 6 shows the efficacy of the Example Composition 2 during the period of 1-4 weeks, 4-6 weeks, 8 weeks, 3 months, 6 months, 1 year, or over 1 year from the administration of the Example Composition 2 . . . . The subjects administered with the Example Composition 2 observed a significant reduction or completely clear in the appearance of their acne.

At 1-4 weeks 40 out of 42 subjects showed improvement in their acne symptoms. Thus, 95% of the subjects showed improvement within 4 weeks from administering the Example Composition 2.

Up to 8 weeks 42 out of 42 subjects showed improvement in their acne symptoms. Thus, 100% of the subjects showed improvement within 8 weeks from administering the Example Composition 2.

Up to 3 months 42 out of 42 subjects showed improvement or significant improvement or completely clear in their acne symptoms. Thus, 100% of the subjects showed improvement or significant improvement or completely clear within 3 months from administering the Example Composition 2.

### Summary of the Results

The subjects described in Example 2 were administered with a less number of tablets per day of the Example composition 2 as 4 tablets or less per day, whereas the subjects described in Example 1 were administered with 6 dosages per day of the Example Composition 1 for the period 1-4 weeks, 4-6 weeks, 8 weeks, 3 months, 6 months, 1 year, or over 1 year from the administration.

The efficacy of the Example composition 2 is significantly superior to that of the Example composition 1. 95% of the subjects administered with the Example composition 2 showed improvement in acne symptoms within 4 weeks from taking the Example composition 2, whereas 55% of the subjects administered with the Example composition 1 showed improvement in acne symptoms within 4 weeks from taking the Example composition 2.

Even at the lower dose such as administration of 1 tablet per day or 2 tablets per day, certain subjects administered with the Example composition 2 experienced in improvement of their acne symptoms within 4 weeks.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A composition for the treatment of acne comprising:
pantothenic acid, or a pharmaceutically acceptable derivative, salt or prodrug thereof;
folic acid;
nicotinamide;
a source of copper;
a source of zinc; and
Urtica dioica.

2. A composition according to claim 1, wherein the pantothenic acid, or a pharmaceutically acceptable derivative, salt or prodrug thereof, is selected from the group consisting of pantothenic acid, calcium pantothenate and pantothenol.

3. A composition according to claim 2, wherein the pantothenic acid, or a pharmaceutically acceptable derivative, salt or prodrug thereof, is calcium pantothenate.

4. A composition according to any one of claims 1 to 3, wherein the source of copper is selected from the group consisting of copper gluconate, copper sulphate, copper acetate and copper citrate.

5. A composition according to claim 4, wherein the source of copper is copper gluconate.

6. A composition according to any one of claims 1 to 5, wherein the source of zinc is selected from the group consisting of zinc gluconate, zinc sulphate, zinc acetate and zinc citrate.

7. A composition according to claim 6, wherein the source of zinc is zinc gluconate.

8. A composition according to any one of claims 1 to 7, wherein the Urtica dioica is Urtica dioica extract, Urtica dioica root extract or Urtica dioica root extract dry concentrate.

9. A composition according to any one of claims 1 to 8, wherein the pantothenic acid, or a pharmaceutically acceptable derivative, salt or prodrug thereof, is present in an amount sufficient to provide an amount of pantothenic acid of from about 1% to about 60% by weight of the composition.

10. A composition according to any one of claims 1 to 9, wherein Urtica dioica is present in an amount of from about 5% to about 70% by weight of the composition.

11. A composition according to any one of claims 1 to 10, wherein Urtica dioica is present in an amount of from about 5% to about 20%, about 20% to about 30%, or about 30% to about 65% by weight of the composition.

12. A composition according to any one of claims 1 to 11 comprising any one or more of the following:
folic acid present in an amount of from about 0.001% to about 0.1% by weight of the composition;
nicotinamide present in an amount of from about 3% to about 15% by weight of the composition;
copper present in an amount of from about 0.001% to about 0.15% by weight of the composition; and
zinc present in an amount of from about 0.05% to about 2% by weight of the composition.

13. A composition according to any one of claims 1 to 12, further comprising any one or more of the following:
biotin;
a form of vitamin A; and
a source of silicon.

14. A composition according to claim 13, comprising any one or more of the following:
biotin present in an amount of from about 0.001% to about 0.1% by weight of the composition;
vitamin A present in an amount of from about 0.001% to about 0.5% by weight of the composition; and
silicon present in an amount of from about 0.1% to about 3% by weight of the composition.

15. A composition according to any one of claims 1 to 8, wherein the pantothenic acid, or a pharmaceutically acceptable derivative, salt or prodrug thereof, is present in an amount sufficient to provide an amount of pantothenic acid of from about 200 mg to about 700 mg.

16. A composition according to any one of claims 1 to 8, wherein the Urtica dioica is present from about 50 mg to about 3,000 mg.

17. A composition according to any one of claims 1 to 8, wherein the Urtica dioica is present from about 90 mg to about 2,000 mg.

18. A composition according to any one of claims 1 to 8, wherein the Urtica dioica is present about 1,500 mg.

19. A composition according to any one of claims 1 to 8 comprising any one or more of the following:
folic acid present in an amount of from about 50 μg to about 150 μg;
nicotinamide present in an amount of from about 100 mg to about 300 mg;
copper present in an amount of from about 200 μg to about 350 μg; and
zinc present in an amount of from about 1 mg to about 20 mg.

20. A composition according to any one of claims 15 to 19, further comprising any one or more of the following:
biotin;
a form of Vitamin A; and
a source of silicon.

21. A composition according to claim 20, comprising any one or more of the following:
biotin present in an amount of from about 300 μg to about 700 μg;
vitamin A present in an amount of from about 50 μg to about 1200 μg; and
silicon present in an amount of from about 1 mg to about 50 mg.

22. A composition according to any one of claim 13, 14, 20 or 21, wherein the vitamin A is present as Vitamin A acetate.

23. A composition according to any one of claim 13, 14, 20 or 21, wherein the source of silicon is colloidal anhydrous silica.

24. A composition for the treatment of acne comprising the following:

| Component | Amount (weight %) |
|---|---|
| Biotin | 0.01-0.03 |
| Calcium pantothenate | 10-25 |
| Folic acid | 0.003-0.01 |
| Vitamin A acetate | 0.03-0.1 |
| Silicon | 0.30-0.80 |
| Copper gluconate | 0.05-0.20 |
| Nicotinamide | 5-10 |
| Zinc gluconate | 1.5-3.0 |
| Urtica dioica | 40-70 |

25. A composition for the treatment of acne comprising the following:

| Component | Amount | |
|---|---|---|
| Biotin | 450-550 | μg |
| Calcium pantothenate | 500-600 | mg |
| Folic acid | 110-150 | μg |
| Vitamin A acetate | 150-250 | μg |
| Silicon | 10-15 | mg |
| Copper gluconate | 1-3 | mg |
| Nicotinamide | 150-200 | mg |
| Zinc gluconate | 50-60 | mg |
| Urtica dioica | 1,450-1,550 | mg |

26. A composition for the treatment of acne comprising the following:

| Component | Amount (weight %) |
|---|---|
| Biotin | 0.03-0.06 |
| Calcium pantothenate | 35-65 |
| Folic acid | 0.005-0.02 |
| Vitamin A acetate | 0.01-0.05 |
| Silicon | 0.30-3.0 |
| Copper gluconate | 0.1-0.40 |
| Nicotinamide | 10-20 |
| Zinc gluconate | 3-7.0 |
| Urtica dioica root Extract | 5-15 |

27. A composition for the treatment of acne comprising the following:

| Component | Amount | |
|---|---|---|
| Biotin | 450-550 | μg |
| Calcium pantothenate | 500-600 | mg |
| Folic acid | 110-150 | μg |
| Vitamin A acetate | 150-250 | μg |
| Silicon | 10-15 | mg |
| Copper gluconate | 1-3 | mg |
| Nicotinamide | 150-200 | mg |
| Zinc gluconate | 50-60 | mg |
| Urtica dioica root Extract | 50-150 | mg |

28. A composition according to any one of claims 1 to 27, further comprising one or more optional excipients.

29. A composition according to any one of claims 1 to 28, wherein the composition is a dosage form selected from the group consisting of a tablet, caplet, capsule, gel cap, pellet and granule.

30. A composition according to claim 29, wherein the dosage form is a tablet.

31. A composition according to claim 30, wherein the tablet is a coated tablet.

32. A composition according to claim 30 or claim 31, wherein the tablet weighs from about 1000 mg to about 2500 mg.

33. A composition according to any one of claims 29 to 32, wherein the dosage form is an oral dosage form.

34. A method of reducing, treating and/or preventing acne and/or related skin disorders comprising the administration of a composition according to any one of claims 1 to 33, to a subject in need of such treatment.

35. A method of reducing, treating and/or preventing the appearance of acne and/or related skin disorders on the skin comprising the administration of a composition according to any one of claims 1 to 33, to a subject in need of such treatment.

36. A method according to claim 34 or claim 35, wherein the method comprises administration of the composition to the subject orally.

37. A method according to claim 36, wherein the administration provides to the subject Urtica dioica in an amount of between about 0.7 g and about 7.5 g per day.

38. A method according to claim 37, wherein the administration provides to the subject Urtica dioica in an amount of about 6 g per day.

39. A method according to claim 37, wherein the administration provides to the subject Urtica dioica in an amount of about 1.5 g per day.

40. A method according to claim 37, wherein the administration provides to the subject Urtica dioica in an amount of about 3 g per day.

41. A method according to claim 37, wherein the administration provides to the subject Urtica dioica in an amount of about 0.75 g per day.

42. Use of a composition according to any one of claims 1 to 33, in the manufacture of a medicament for the reduction, treatment and/or prevention of acne and/or related skin disorders.

43. Use of a composition according to any one of claims 1 to 33, in the manufacture of a medicament for the reduction, treatment and/or prevention of the appearance of acne and/or related skin disorders on the skin.

44. A composition according to any one of claims 1 to 33, when used for the reduction, treatment and/or prevention of acne and/or related skin disorders.

45. A composition according to any one of claims 1 to 33, when used for the reduction, treatment and/or prevention of the appearance of acne and/or related skin disorders on the skin.
